(19) United States
(12) Patent Application Publication
(10) Pub. No.: US 2013/0330413 A1
(43) Pub. Date: Dec. 12, 2013

(54) NUTRITIONAL COMPOSITIONS COMPRISING CHITIN MICROPARTICLES

(71) Applicant: Neill Moray MacKenzie, Hertfordshire (GB)

(72) Inventor: Neill Moray MacKenzie, Hertfordshire (GB)

(73) Assignee: CMP THERAPEUTICS LIMITED, Hertfordshire (GB)

(21) Appl. No.: 13/816,563

(22) PCT Filed: Aug. 17, 2011
(86) PCT No.: PCT/GB2011/001251

§ 371 Date: Jul. 25, 2013

Related U.S. Application Data
(60) Provisional application No. 61/374,472, filed on Aug. 17, 2010.

(51) Int. Cl. A61K 31/722; A23L 1/30

(52) U.S. Cl. CPC A61K 31/722; A23L 1/30
USPC 424/499; 424/278.1

(57) ABSTRACT

Nutritional oral compositions are disclosed that contain a nutritional component, such as a macronutrient or a micronutrient. The nutritional compositions also include a chitin microparticle preparation preferably obtained by microfluidisation, wherein the chitin microparticles have an average diameter of between 1 and 100 μm.

### FIELD OF THE INVENTION

The present invention relates to nutritional compositions, in particular compositions containing chitin microparticles, which may be used to promote up-regulation of the cell mediated immune system and to prevent or alleviate conditions that would benefit from an up-regulation of a Th1 response or regulatory immune response.

### BACKGROUND OF THE INVENTION

There is a continuing need to improve ways of ensuring a healthy immune system in order to protect the body from microbes and pathogens. Macrophages play a key role in the innate immune system by promoting phagocytic clearance and the secretion of cytokines that promote an effective cell mediated immune response to particulates including microbes and pathogens. The principle cytokines produced during phagocytosis are IL-12, TNF-α, and IL-18. These macrophage cytokines subsequently induce IFN-γ production by NK cells and Th1 lymphocytes. IFN-γ acts synergistically with these cytokines to promote a Th1 cell mediated immune response and also down-regulate the production of Th2 cytokines, in particular IL-4, IL-5 and IL-13 which are strong mediators of allergy.

Studies by Shibata et al (1-4) have shown that oral delivery of 1-10 μm phagocytosable particulate chitin obtained by sonication, centrifugation and sieving results in an elevation of Th1 cytokines in mouse spleen cell cultures. The effect was specific to the particulates as no elevation was produced by soluble chitin. It could also be reproduced in 1 μm polystyrene microspheres coated with N-Acetyl-D-Glucosamine, which is the main component of chitin. It was also demonstrated that oral administration of chitin down-regulates serum IgE and lung eosinophilia in a murine model of ragweed allergy (1).

Shibata et al have also developed a mouse model of allergic airway inflammation and orally administered chitin preparations to the mice (Shibata 2000). Ragweed-specific IgE levels were significantly reduced after daily oral administration of chitin to ragweed-sensitised mice, before and during immunisation.

When chitin was administered prophylactically to mice who were subsequently administered ragweed, IL-4, IL-5 and IL-10 production was significantly reduced and low but significant levels of IFN-γ were detected.

Sonicated particulate chitin also has a prophylactic effect when administered to C57BL6 mice, which are higher responders for cell-mediated immunity/Th1 responses, but lower responders for allergic responses compared with BALE/c mice. When ragweed-sensitised mice were treated simultaneously with ragweed and chitin, the levels of IL-4, IL-5 and IL-10 produced were significantly reduced compared to those stimulated by ragweed alone.

In our earlier application, WO 03/015744, we described experiments in mice which demonstrated that a suspension of CMP in saline administered intranasally has beneficial immune modulating properties, which can be applied for the treatment of allergic disease and can enhance protection by up-regulation of mechanisms of innate immunity against viral and bacterial infections of the respiratory tract. The beneficial immune regulating properties can also be applied for the treatment of conditions that would benefit from an up-regulation of natural killer (NK) cell activity and/or the secretion of interferon-γ (IFN-γ), such as the treatment of cancer.

In our earlier application, WO 07/148048, we described the use of CMP as an adjuvant in vaccine compositions. In particular, CMP compositions were found to be capable of synergistically enhancing the protection raised against an antigen from an infectious agent when the CMP compositions were combined with a further adjuvant, such as the cholera toxin B subunit (CTB).

WO 09/142988 discloses the use of CMP as an adjuvant to enhance the protective immunity against infectious diseases such as Listeria. In particular, the chitin microparticles are used as an adjuvant in combination with a cholesterol lowering agent.

### SUMMARY OF THE INVENTION

It would be advantageous to be able to boost the innate immune response in a subject before allergy is diagnosed or even before symptoms develop or become severe. Therefore, the present invention seeks to provide a product that can achieve this.

Broadly, the present invention provides an oral nutritional composition, for oral consumption and optionally for enteral adsorption, wherein the nutritional composition includes a chitin microparticle preparation (CMP). Typically, the chitin microparticles have an average diameter of between 1 and 100 μm, and more preferably between 1 and 20 μm and/or and are obtainable by microfluidisation.

Accordingly, in a first aspect, the present invention provides an oral nutritional food composition, e.g. for enteral adsorption, the composition having one or more nutritional components and a chitin microparticle preparation (CMP), wherein the chitin microparticles have an average diameter of between 1 and 100 μm and are obtainable by microfluidisation.

In a further aspect, the present invention provides an oral nutritional composition according to the present invention for use in a method of treating allergy, wherein the composition comprises a chitin microparticle preparation (CMP), wherein the chitin microparticles have an average diameter of between 1 and 100 μm and are obtainable by microfluidisation.

In a further aspect, the present invention provides the use of a chitin microparticle preparation (CMP) according to the present invention in the preparation of an oral nutritional composition, the composition having one or more nutritional components and the chitin microparticle preparation (CMP), wherein the chitin microparticles have an average diameter of between 1 and 100 μm and are obtainable by microfluidisation.

In a further aspect, the present invention provides a foodstuff for oral consumption, the foodstuff including a nutritional composition with one or more nutritional components and a chitin microparticle preparation (CMP) as described herein, wherein the chitin microparticles have an average diameter of between 1 and 100 μm and are obtainable by microfluidisation.

As noted above, the compositions of the present invention may be orally administered. In this connection, the term “administered” and/or “administration” preferably refers to oral ingestion, intake and/or consumption by the subject. Preferably, the composition is consumed by eating and/or drinking. In another embodiment, the composition is administered by tube feeding.

If the nutritional compositions are formulated to be administered orally, the compositions may be a liquid oral nutritional supplement (e.g., incomplete feeding) or a complete feeding. In this manner, the nutritional compositions may be administered in any known form including, for example, tablets, capsules, liquids, chewables, soft gels, sachets, powders, syrups, liquid suspensions, emulsions and solutions in convenient dosage forms.

“A nutritional composition may be a food product intended for human consumption, for example, a beverage, a drink, a bar, a snack, an ice cream, a dairy product, for example a chilled or a shelf-stable dairy product, a fermented dairy product, a drink, for example a milk-based drink, an infant formula, a growing-up milk, a confectionery product, a chocolate, a cereal product such as a breakfast cereal, a sauce, a soup, an instant drink, a frozen product intended for consumption after heating in a microwave or an oven, a ready-to-eat product, a fast food or a nutritional formula.

A nutritional formula encompasses any nutritionally complete or supplementary formulation (a nutritional supplement, for example). As used herein, “nutritionally complete” are preferably nutritional products that contain sufficient types and levels of macronutrients (protein, fats and carbohydrates) and micronutrients to be sufficient to be a sole source of nutrition for the subject to which it is being administered to. Patients can receive 100% of their nutritional requirements from such complete nutritional compositions. According to one embodiment, the nutritional formula is a supplementary formulation providing supplementary nutrition. A “supplementary formula” may not be nutritionally complete, but preferably contains specific nutrients that are supportive, for example in combination with physical exercise, with further of the beneficial effects of the invention, and/or which address specific or additional needs of the subject.

The nutritional formula may be a generally applicable nutritional formula, for example adapted to subjects of a specific age, for example a formula for children, but it may also be a formula for elderly patients, for intensive care patients, or a specially adapted formula for patients suffering from a specific disease, for example. Any nutritional formula may be reconstitutable, that is, present in a substantially dried, for example powdered form, or ready-to-drink, in the form of liquid formulas, for example.

The nutritional composition of the present invention provides an orally consumable dose of CMP that may be regularly taken to boost the immune system of an individual over an extended period of time. In this way, the immune system of the individual may respond better with microbes, pathogens and allergens that enter the individual's body. As a result, there may be an increase in the general health of the individual with none, fewer or less severe symptoms of a particular affliction.

The specific and non-specific immune system may thus be boosted. However, a particular advantage of the present composition is that the composition may be used without an allergen so that the composition provides an increase in the non-specific immune system of an individual. Of course, an allergen may be used in conjunction with the nutritional composition in order to improve the specific immune system for that allergen. As a further advantage, the nutritional composition may form part of the individual's regular nutritional intake, for example as a daily dietary supplement. As a further advantage, the CMP may be more readily absorbed into the system by enteral with the nutritional component.

Accordingly, the nutritional composition may be taken in additional to an individual's normal nutritional intake, for example as a dietary supplement. Alternatively, the nutritional composition may form a substantial proportion of the individual's nutritional intake, for example, as infant formula (for infant human individuals) or as animal food (for animal individuals).

The nutritional component may be one or more macronutrients. Macronutrients include protein, carbohydrate and lipid. The nutritional composition contains preferably two and more preferably all three of the group of protein, carbohydrate and lipid macronutrients.

Additionally or alternatively, one or more of the nutritional components may be a micronutrient. Micronutrients include vitamins, minerals and salts. When one or more of the nutritional components is a micronutrient, the micronutrients are preferably present in a number of different vitamins and/or minerals so that the composition provides a broad spectrum of micronutrients.

Suitable macro- and micro-nutrients are known in the art and the selection of the nutritional composition will depend on the nature of the nutritional composition. The skilled person would be able to select nutritional components from those known to suit the needs of any particular nutritional composition. Typical proteins include whey protein, casein, milk-derived proteins and soy-derived proteins. Typical carbohydrates include lactose, maxtodextrin, fructose, glucose, starch and saccharose. Typical lipids include palm olein, linoleic acid, α-linolenic acid, high oleic sunflower oil, high oleic safflower oil and oils containing arachidonic acid and/or docosahexaenoic acid.

A range of vitamins and/or minerals may be included in the food composition such as vitamin A, vitamin B1, vitamin B2, vitamin B6, vitamin B12, vitamin C, vitamin D, vitamin K, folic acid, inositol, niacin, biotin, panthothenic acid, choline, calcium, phosphorus, iodine, iron, magnesium, copper, zinc, manganese, chloride, potassium, sodium, selenium, chromium, molybdenum, taurine nad L-carnitine. Minerals are typically added in salt form.

The nutritional composition may be in any form, such as a liquid suspension, semi-liquid, solid, powder, gum or tablet form. The composition may be stored in powder form and mixed with another substance before consumption. Typically, powdered nutritional compositions are mixed with water to produce a nutritional drink. The CMP in the nutritional drink will be a suspension, but the nutritional component or components may be dissolved or in a suspension. The water used to make the nutritional drink may also contain nutritional (or otherwise) particles or solutes prior to mixing with the nutritional composition.

The nutritional composition may be a human milk fortifier, an infant formula, a follow on formula, a growing up milk, a protein formula, a sport recovery formula, a sport energy formula or a sport electrolyte formula. The nutritional composition may be a constituent of an infant cereal, a baby food, a yogurt, a cereal bar, a breakfast cereal, a dessert, a beverage, a confectionary item, a frozen food, a soup or an animal food. Preferably, the composition is or is included in infant formula.

The amount of CMP in each nutritional composition will depend on how much and how often the composition is to be consumed by an individual. Typically, the nutritional composition includes up to 100 mg of CMP per recommended daily allowance of the nutritional composition. This may be consumed in a single intake or as a series of intakes, for example, an intake of the nutritional composition with each meal of the day. Preferably, the nutritional composition includes between 1 and 100 mg, more preferably between 20 and 80 mg and more preferably between 40 and 60 mg per recommended daily allowance of the nutritional composition. The amount of CMP in the nutritional composition may depend on the body weight of the individual consuming the nutritional composition. The compositions preferably provide between about 0.01 and 100 mg of active compound per kg of body weight of an individual, and more preferably between about 0.5 and 10 mg/kg of body weight.

The average diameter of the microparticles may be measured in a number of ways, including by laser diffraction or light obscuration. As understood by the skilled person, the use of different techniques may result in variation in the recorded average diameter size of the microparticles. For example, one technique may give particle size as a sphere of the minimum length of a particle, whereas another technique may give particle size as the maximum length of a particle and so for an irregularly shaped particle, the two measurements will differ.

Preferably, the chitin microparticles have an average diameter based on a sphere of minimum length of less than 50 μm, more preferably less than 40 μm, still more preferably less than 20 μm, more preferably less than 10 μm and most preferably less than 5 μm.

Preferably the chitin microparticles have an average diameter based on a sphere of maximum length of less than 100 μm. More preferably the chitin microparticles have an average diameter based on a sphere of maximum length of less than 80 μm, more preferably less than 70 μm and more preferably less than 60 μm.

Average particle size is preferably less than 10 μm if measured by light obscuration, for example using an Accusizer™. Average particle size is preferably between 40 and 60 μm if measured by laser diffraction. Other techniques for measuring particle size may be used.

As we have found that the effects caused by chitin microparticles are size dependent, it is preferred that the chitin microparticles have average diameters based on a sphere of minimum length which are 10 μm or less than 10 μm. An upper limit of chitin particles size may be functionally defined by macrophages not recognising the particles. The lower size limit is less important, but preferably the particles are at least 1 μm in diameter. The lower size limit is functionally defined by the chitin particles becoming soluble and hence also not being recognised by macrophages. Particles size and size distribution can readily be determined by the skilled person for example using flow cytometry or a microscope. Alternatively or additionally, the chitin microparticles can be made by coating carrier particles, e.g. formed from a biocompatible material such as polystyrene or latex, with N-Acetyl-D-Glucosamine, chitin or a fragment thereof, to form particles having the sizes as defined above, and these compositions are included within the term chitin microparticle composition as used herein.

It should be recognised that in a composition, the chitin microparticles will have a distribution of sizes, typically a normal distribution, and that not all particles within a population will necessarily meet these size limits. However, within a population of chitin microparticles forming a CMP preparation, preferably at least 60%, more preferably at least 75%, more preferably at least 90%, and more preferably 95% and most preferably at least 99%, of the chitin particles have a size distribution within the limits set out above.

Preferably chitin is produced by physically reducing it, e.g. by sonication or milling. In a preferred embodiment, the particles produced from a microfluidising instrument, such as the method described in our earlier patent application WO 2008/053192. Particle shape is not limited. Sonication will typically produce “boulder-shaped” particles that are essentially spheroid in nature but with a varying degree of deviation from a sphere. In other words, the particle is a spheroid with angular edges.

However, the present invention has found that the shape of the chitin microparticles obtainable from the microfluidiser method differ from those produced from sonication. When produced from a microfluidiser method, the particles are “fluffy”. As a result, the particles have a high surface area. Such “fluffy” chitin microparticles are more stable in suspension than the angular spheroid chitin microparticles produced by techniques such as sonication or milling and thus result in a more stable chitin microparticle preparation. The skilled person will be able to measure the stability of chitin microparticle compositions obtainable by microfluidisation, for example by determining whether a composition is capable of forming a stable aqueous suspension at a concentration of 5 mg/ml and a temperature of 25° C. for at least one hour. This may be contrasted with compositions produced by sieving, sonication or milling which tends to fall out of suspension and sediment at the bottom of their container in a short period of time, e.g. in less than 10 minutes. An exemplary chitin microparticles preparation was prepared using chitin microparticles obtained from the microfluidiser method. This composition was stable in solution for several weeks. In this way, the chitin microparticles obtained from the microfluidiser method are particularly suitable for use in nutritional composition of the present invention, such as a yogurt drink. Alternatively or additionally, the skilled person will be able to recognise that that the chitin microparticles obtainable by microfluidisation differ from particles produced by techniques such as milling in that they the microfluidisation manufacturing procedures that produces compositions that have a microgel or gel-like consistency in an aqueous composition, and that the gel compositions may then be dried to produce a powder.

As well as possessing different physical properties, the experiments reported herein demonstrate that the chitin microparticle compositions obtainable by microfluidisation has enhanced biological effects as compared to corresponding compositions produced by techniques such as sonication or milling, in particular enhancing the secretion of IFN-γ levels produced by white blood cells from an allergic individual. Preferably, chitin microparticle compositions obtainable by microfluidisation are capable of producing at least 2-times, more preferably at least 3-time, and more preferably at least 4-times, the IFN-γ response in human white blood cells as compared to chitin preparations obtained by milling or sonication.

Chitin is a polymer of N-acetyl-D-glucosamine and has a similar structure to cellulose. It is an abundant polysaccharide in nature, comprising the horny substance in the exoskeletons of crab, shrimp, lobster, cuttlefish, and insects as well as fungi. Any of these or other sources of chitin are suitable for the preparation of CMP preparations for use according to the present invention. A small degree of deacetylation of chitin may occur during the processing of the chitin. However, no more than 50% deacetylation may be tolerated, preferably no more than 40%, more preferably no more than 30%, more preferably no more than 20% and most preferably no more than 10% deacetylation. At levels greater than 50% deacetylation, chitosan (a deacetylated polymer of glucosamine) is formed.

In generally, the chitin microparticle compositions are employed in accordance with the present invention in combination with nutritional components of the food or drink so that the immune system of an individual consuming the food or drink is boosted, helping to prevent or treat allergy. However, in some alternative embodiments, the composition may includes an allergen. These compositions can be employed in the treatment of allergies and allergic symptoms, such as anaphylactic shock, which are associated with conventional desensitisation therapy. Oral application of IL-12 has been shown to suppress anaphylactic reactions and so administering an allergen with a CMP composition in a nutritional composition should help to moderate the anaphylactic reactions arising during desensitisation therapy designed to build up tolerance to an allergen. Allergens can be readily extracted from food and are commercially available as they are used in the diagnosis and treatment of allergy. Whether or not an allergen is included in the composition, the present invention is particularly applicable to the treatment of food allergies for example those involving common food allergens such as milk, wheat, gluten, eggs, nuts or shellfish. The skilled person will be able to formulate these with the CMP composition for consumption by an individual.

The present invention may be used to up-regulate the cell-mediated immune system and so in another aspect the present invention provides the use of the nutritional composition described herein to help to prevent, treat or alleviate symptoms of a number of conditions associated with up-regulation of the cell-mediated immune system. Conditions that benefit from the up-regulation of the cell-mediated immune system include the treatment or prophylaxis of microbial infections, including bacterial infections, fungal infections and viral infections, particularly among vulnerable patient groups such as the elderly, premature babies, infants, transplantation patients, immunosuppressed patients such as chemotherapy patients, hospital patients at risk of opportunistic infection, patients on ventilators, cystic fibrosis patients and patients with AIDS. The invention is particularly applicable to the treatment of ear, nose, throat and lung infections.

Specific examples of bacterial infection include the treatment of infection by microorganisms such as Pseudomonas aeruginosa, Streptococcus species such as Streptococcus pneumoniae, Streptococcus pyrogenes, Streptococcus agalactiae, Haemophilus influenza, Klebsiella pneumoniae, Yersinia enteocolitica, Salmonella, Listeria, Mycobacterial infections including Mycobacterium tuberculosis, Mycobacterium leprae, parasitic infections including Leishmania species and Schistosoma species.

One condition caused by microbial infection, typically by Streptococcus pneumoniae, is recurrent ear infections such as Otitis media. These conditions occur in children and adults and are currently treated using antibiotics. It would be advantageous to use the chitin microparticle compositions of the invention to prevent or treat these conditions and reduce the need for antibiotics.

The preparations of the invention can be used in the treatment of tuberculosis either to treat an existing infection or to protect vulnerable patient groups from infection.

Other examples of microbial infections include bacterial pneumonias, such as ventilator-associated pneumonia, and cystic fibrosis associated infections.

Examples of fungal infections include fungal infections such as invasive pulmonary aspergillosis and invasive pulmonary candidiasis, Pneumocystis carinii pneumonia, Coccidioides and Crytococcus infections, e.g. in immunosuppressed patients.

Examples of viral conditions treatable according to the present invention include pulmonary viral infections such as respiratory syncytial virus bronchiolitis, especially in infants and the elderly, or influenza virus, or rhino virus. Numerous studies have shown that during the progression of AIDS, mononuclear cells lose their ability to secrete IL-2, IL-12 and IFN-γ and produce increased levels of IL-4, which allows the HIV virus to proliferate. Therefore treatment with CMP, given in a nutritional composition will be useful in reducing the progression of HIV infection by restoring IL-12 and IFN-γ levels.

Furthermore, the nutritional composition of the present invention may be used to help to prevent, treat or alleviate symptoms of a gastrointestinal disorder such as inflammatory bowel disease, Crohn's disease, ulcerative colitis, inflammatory bowel disorder, irritable bowel syndrome, irritable bowel syndrome-diarrhea, irritable bowel syndrome-constipation, irritable bowel syndrome-alternating, irritable bowel syndrome-mixed, dyspepsia, gastro-esophageal reflux, diverticulitis, diverticular disease, gastroparesis, microscopic colitis, lymphocytic colitis, collagenous colitis, indeterminant colitis, eosinophilic esophagitis, HIV-associated diarrhea, pseudo-membranous colitis, diarrhea associated with immunodeficiency disorders, small bowel overgrowth syndrome, celiac disease, Whipple's disease, CMV-associated colitis, Behcet's syndrome and combinations thereof. In particular, the nutritional composition may be used to prevent, treat or alleviate symptoms of Crohn's disease.

In addition to chitin microparticles, the CMP preparations may comprise one or more of an acceptable excipient, carrier, propellant, buffer, stabiliser, isotonicizing agent, preservative or anti-oxidant, flavouring or other materials well known to those skilled in the art. Such materials should be non-toxic and should not interfere with the efficacy of the chitin microparticles.

Preservatives may be included in the nutritional compositions to extend shelf life of the compositions, for example, by retarding microbial growth, in order to allow multiple use packaging. Examples of preservatives include calcium propionate, sodium nitrate, sodium nitrite, sulfites (sulfur dioxide, sodium bisulfite, potassium hydrogen sulfite, etc.), disodium EDTA, butylated hydroxyanisole (BHA) and butylated hydroxytoluene (BHT). Preservatives are typically employed in the range of about 0.1% to 1.0% (w/v).

Preferably, the nutritional compositions are provided with “prophylactically effective amount” or a “therapeutically effective amount” (as the case may be, although prophylaxis may be considered therapy) for an individual, this being sufficient to show benefit to the individual, e.g. providing alleviation of allergy or another condition or prophylaxis for an acceptable period. Typically, this will be to cause a therapeutically useful activity providing benefit to the individual. The compositions preferably provide between about 0.01 and 100 mg of active compound per kg of body weight of an individual, and more preferably between about 0.5 and 10 mg/kg of body weight. By way of example, this could be achieved using an infant formula to provide approximately 1.25 mg of CMP particles per 25 g portion of powdered formula (to make approximately 150 ml of reconstituted formula).

In another aspect, the present invention provides a foodstuff including the nutritional composition as described herein. A foodstuff contains one or more food nutrients arising from processed or unprocessed food materials, such as fruit, vegetable, seeds, beans, pulses, dairy products, meat and other animal-derived products. Thus the present invention may result from adding a nutritional component and a CMP to a conventional foodstuff, such as confectionary or a yogurt drink. By adding the CMP to a foodstuff, the chitin microparticles may be consumed as a part of the consumer's usual daily meal or snack.

The CMP and nutritional component of the nutritional composition may be added to the food nutrient either together or separately. If added separately, the CMP and nutritional component should be added within the same food processing process. In other words, the CMP and nutritional component should be added in the same production line or location.

The optional and preferred features of one aspect of the invention may be applied to the other aspects of the invention and vice versa. Embodiments of the present invention will now be described by way of example and not limitation.

The present invention includes the combination of the aspects and preferred features described except where such a combination is clearly impermissible or is stated to be expressly avoided. Embodiments of the present invention will now be described by way of example and not limitation with reference to the accompanying figures.

### BRIEF DESCRIPTION OF THE FIGURES

FIG. 1 shows the results of experiments in which orally administered CMP compositions were tested for efficacy in preventing and managing allergic symptoms.

FIG. 2 shows the effect of CMP compositions on cytokine secretion by white blood cells from an allergic individual in an in vitro study.

### DETAILED DESCRIPTION

### Materials and Methods

### Chitin Microparticle Suspension Preparation (CMP)

Chitin microparticles were prepared from purified chitin (Sigma-Aldrich, Poole, UK) by sonication of a suspension of 10 mg/ml in endotoxin free PBS at maximum output for 20 min with cooling on ice every 5 min. The slurry was centrifuged at 1000×g for 10 min to remove large particles and the microparticles were collected by centrifugation at 4000×g and washed 3 times with PBS to remove any solubilized chitin. The supernatant contained a uniform suspension of small particles as judged by light microscopy using a haemocytometer with 50 μm squares and were comparable in size to 1 μm latex spheres (Polysciences, Inc., Warrington, Pa., USA). Particles less than 5 μm in diameter were quantified with a Celltac Hematology Analyser (Nihon Kohden, Inc.). Preparations were found to contain 99.9% microparticles less than 5 μm in diameter and at a concentration in the order of 10¹¹/ml. Endotoxin was measured by Limulus Amebocyte Lysate Assay (BioWhittaker Co,) and shown to be <1 EU/ml. In other embodiments, a CMP suspension was made using microfluidiser as described in WO 2008/053192.

### Infant Formula Preparation

A mixture of 7.0 g protein source (70% whey, 30% casein), 36 g of carbohydrate source (lactose) and 17 g of lipid source (high oleic sunflower oil) were mixed together with warm water (50-80° C.) to form a liquid mixture. The mixture was homogenised and thermally treated in an autoclave to reduce the bacterial content of the mixture. The mixture was allowed to cool and Vitamins A, D, E, K1, C, B1, B2, B6, B12, Niacin and Folic acid (in pg to mg standard amounts), minerals salts containing Na, K, Cl, Ca, P, Mg, Mn, Se, Fe, Cu, Zn and I (in pg to mg standard amounts) and 0.75 ml of a 5 mg/ml suspension of CMP particles (to give 3.75 mg of CMP microparticles in approximately 70 g of formula) were added to the cooled mixture.

The liquid mixture was transferred to a freeze drier in order to dry the mixture to a powder. The powder has a moisture content of less than about 5% by weight. The powder is then vacuum sealed in a plastic container for later reconstitution and consumption.

### Probiotic Yogurt Drink Preparation

Skimmed milk, CMP suspension in water (5 ml of a 5 mg/ml suspension), dextrose, pectin, aspartame, acesulfame K, probiotic and Vitamin K was added to 120 ml of yogurt. The mixture was stirred for 10 minutes to produce a probiotic yogurt drink. The resulting drink was refrigerated ready for consumption.

### Energy Bar

The dry ingredients of maltodextrin (100 g), oat bran (200 g), puffed rice (60 g) milk protein isolate (100 g), crystalline fructose (80 g), mineral premix (30 g), rice flour (60 g) were mixed together. To this dry mixture a warm mixture of golden syrup (340 g), butter (40 g), flavourings (10 g) and CMP suspension (20 ml of 25 mg/ml water suspension) was added slowly with mixing. The resulting admixture was rolled and pressed into a slab and cut into 50 g bars for packaging.

### Dog Food

The following Pet Food grade ingredients were mixed to prepare a canine food mixture: corn starch (650 g); soy protein (250 g); calcium carbonate (20 g); cellulose (22 g); coconut oil (17 g); dicalcium phosphate (12 g); aqueous CMP suspension (5 ml of 5 mg/ml suspension); choline chloride (2.5 g); magnesium oxide (2 g); sodium chloride (1 g); vitamins D3, E and B12; riboflavin and folic acid.

### In Vivo Study

The effect of oral treatment with CMP compositions was tested in an OVA food allergy animal model to determine whether CMP compositions have a preventative effect in reducing the risk of developing allergic symptoms and to determine whether CMP compositions are useful for the treatment of allergic symptoms when administered after sensitization has occurred. Six weeks old adult conventional BALB/c mice were sensitized by the oral route—3 applications in the first week and then at weekly intervals with 20 mg of Ovalbumin (OVA) plus 10 μg/mouse of Cholera toxin (used as adjuvant) during 7 weeks. One week after the last sensitization, an oral challenge via gavage with 100 mg of OVA was performed. On the day of the challenge, mice were starved for 2 hours before challenge. Thirty minutes after the challenge, the mice were individually observed during 30 min. Clinical symptoms were recorded and quantified as follows (Allergic Score): 0) no symptoms, less than 4 episodes of scratching; 1) 4-10 episodes of scratching around the nose and head, no diarrhoea; 2) more than 10 episodes of scratching or soft stool; 3) diarrhoea or laboured respiration or cyanosis or the presence of two or more symptoms (scratching and soft stool); 4) diarrhoea in combination with immobility after prodding, bristled fur, laboured respiration or cyanosis; 5) anaphylaxis.

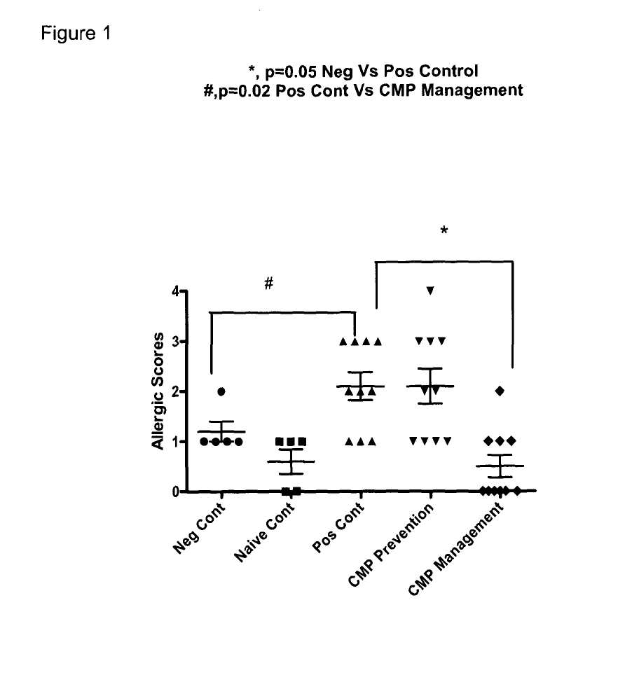

Four hours after the challenge, the mice were sacrificed. The results are shown in FIG. 1 which shows that CMP compositions have a beneficial effect in the management of allergic symptoms in sensitized mice.

### In Vitro Human Cell Study

An in vitro study was carried out to characterise the effect of chitin microparticles on whole blood cells taken from atopic individuals. Microfluidised chitin from ten microfluidisation cycles was compared with appropriate controls.

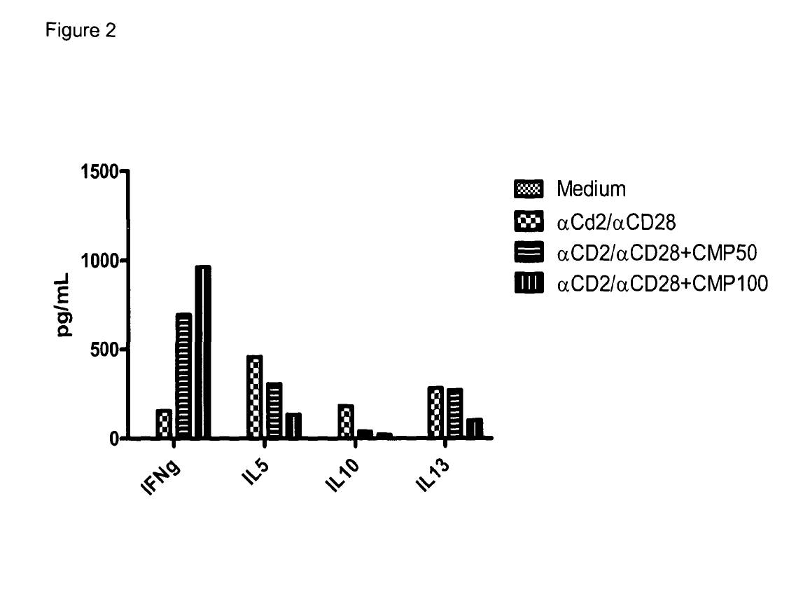

Whole blood cells from an allergic donor (clinical history+SPT to Grass Pollen) were cultured in RPMI complemented with 1% L-glutamine (Sigma), 1% Penicillin/Streptomycin (Sigma), 0.1% Gentamycin (Sigma). Cells were either stimulated with anti-CD2 and anti-CD28 alone or CMP was added at a concentration of either 50 ug or 100 ug along with anti-CD2 and anti-CD28. Unstimulated controls were also added. After 5 days culture supernatants were taken and frozen until further analysis. Human IL-5, IL-10, IL-13, and IFN-γ were measured using a human Th1/Th2 multiplex kit. The results of the study are shown in FIG. 2. This shows that CMP boosts IFN-γ levels in an allergic individual and reduces Th2 cytokines (IL-5, IL-13). The effect is dose dependant.

### REFERENCES

The documents disclosed herein are all expressly incorporated by reference in their entirety.
1. Shibata et al, J. Immunol., 164: 1314-1321, 2000.
2. Shibata et al, J. Immunol., 161: 4283-8, 1998.
3. Shibata et al, Infection and Immunity, 65(5): 1734-1741, 1997.
4. Shibata et al, J. Immunol., 159: 2462-2467, 1997.
5. WO 03/015744
6. WO 07/148048
7. WO 09/142988

1. An oral nutritional composition, the composition comprising one or more nutritional components and a chitin microparticle preparation (CMP), wherein the chitin microparticles have an average diameter of between 1 and 100 μm and are obtainable by microfluidisation.

2. The oral nutritional composition according to claim 1, wherein the chitin microparticles obtainable by microfluidisation have at least one of the following properties:
(a) the chitin microparticles form a stable aqueous suspension at a concentration of 5 mg/ml and a temperature of 25° C. for at least one hour;
(b) the chitin microparticles have a gel-like consistency in aqueous compositions; and
(c) the chitin microparticles have a fluffy shape in contrast to the angular spheroid chitin microparticles produced by sonication.

3. The oral nutritional composition according to claim 1, wherein the composition is a nutritionally complete formulation or a supplementary formulation.

4. The oral nutritional composition according to claim 1, wherein the composition is a nutritional composition for enteral adsorption.

5. The oral nutritional composition according to claim 4, wherein at least one nutritional component is a macronutrient.

6. The oral nutritional composition according to claim 4, wherein the composition includes two or more nutritional components selected from the group of macronutrients of protein, carbohydrate and lipid.

7. The oral nutritional composition according to claim 4, wherein at least one nutritional component is a micronutrient.

8. The oral nutritional composition according to claim 7, wherein the composition includes two or more nutritional components selected from the group of micronutrients of minerals, vitamins and salts.

9. The oral nutritional composition according to claim 1, wherein the composition is a drink.

10. The oral nutritional composition according to claim 1, wherein the composition is a food.

11. The oral nutritional composition according to claim 10, wherein the composition is an infant cereal, a baby food, a yogurt, a cereal bar, a breakfast cereal, a desert, a beverage, a confectionary product, a frozen food, a soup or an animal food.

12. The oral nutritional composition according to claim 1, wherein the composition is a beverage, a drink, a bar, a snack, an ice cream, a dairy product, for example a chilled or a shelf-stable dairy product, a fermented dairy product, a drink, for example a milk-based drink, an infant formula, a growing-up milk, a confectionery product, a chocolate, a cereal product such as a breakfast cereal, a sauce, a soup, an instant drink, a frozen product intended for consumption after heating in a micro-wave or an oven, a ready-to-eat product, a fast food or a nutritional formula.

13. The oral nutritional composition according to claim 1, wherein the chitin microparticles have an average diameter of 20 μm or less.

14. The oral nutritional composition according to claim 1, wherein the chitin microparticles are prepared by reducing the size of particles in a chitin microparticle composition using a microfluidiser.

15.-18. (canceled)

19. A foodstuff for oral consumption, the foodstuff including a nutritional composition with one or more nutritional components and a chitin microparticle preparation (CMP), wherein the chitin microparticles have an average diameter of between 1 and 100 μm and are obtainable by microfluidisation.

20. The foodstuff of claim 19, wherein the foodstuff is an infant cereal, a baby food, a yogurt, a cereal bar, a breakfast cereal, a desert, a beverage, a confectionary product, a frozen food, a soup or an animal food.

21. The oral nutritional composition according to claim 12, wherein said composition is a dairy product, said dairy product being a chilled or a shelf-stable dairy product.

22. The oral nutritional composition according to claim 12, wherein said composition is a drink, said drink being a milk-based drink.

23. A method for the treatment or prevention of allergy, said method comprising administering to an individual in need thereof an effective amount of an oral nutritional composition according to claim 1.

24. A method for the treatment or prevention of conditions benefiting from up-regulation of the cell-mediated immune system, said method comprising administering to an individual in need thereof an effective amount of an oral nutritional composition according to claim 1.
